(19) United States
(12) Patent Application Publication
(10) Pub. No.: US 2026/0007280 A1
(43) Pub. Date: Jan. 8, 2026

(54) Container for automatically measuring the amount and quality of food

(71) Applicant: Pataro Pty Ltd, Strathalbyn (AU)

(72) Inventor: Ben Rolston, Strathalbyn (AU)

(21) Appl. No.: 18/997,022

(22) PCT Filed: Oct. 19, 2023
(86) PCT No.: PCT/AU2023/051044

§ 371 Date: Jan. 18, 2025

(51) Int. Cl. A47J 47/02; B65D 25/20; G01F 23/292; G01N 33/02

(52) U.S. Cl. CPC A47J 47/02; B65D 25/20; G01F 23/292; G01N 33/02; A47J 2203/00; B65D 2203/04; B65D 2203/10

(57) ABSTRACT

A system for allowing a user to automatically measure the contents in a container. The system has a base for holding the contents within the container, a lid for the base of the container, a central processing unit within the container, an antenna connected to the central processing unit for transmitting data. a software application running on a mobile device for communicating with the central processing unit, a remote server for connecting to the central processing unit and to the internet and a sensor device connected to the central processing unit to create data files about the container. The system is characterized in that the sensor device has a particle detector for measuring the contents of the container.

### TECHNICAL FIELD

The present invention relates to containers and, more particularly to a container which can automatically create data about the amount and quality of food within it and communicate this data to a remote user.

### BACKGROUND

People who operate kitchens, including restaurant chefs and household owners, need to know their stock of ingredients to prepare food. When purchasing new ingredients at a shop, it is difficult to accurately know the remaining stock of ingredients at the kitchen. It can be impractical or inconvenient to have to go back to the kitchen to ascertain the stock levels. People often like to purchase ingredients when passing by a shop, rather than having to go home or back to the kitchen to ascertain their stock levels. A restaurant owner may be responsible for the availability of food on a menu, but the owner may not know the levels of the ingredients available in the kitchen to make that food.

Some have attempted to solve this problem by having a live video feed into a refrigerator. However, such technologies do not allow the user to know how much contents are in an opaque container. Therefore, it can be difficult to know how much stock of an ingredient is required to be purchased whilst at a shop or some other location away from the kitchen.

Another means of solving this problem is to conveniently create a shopping list using voice dictation on devices such as Google Home or Amazon Alexa. However, such devices do not let you automatically know the stock levels of ingredients in a kitchen. The assessment of remaining stock levels must be made manually.

It would also be useful if there was a way to automatically and remotely determine whether food within a container has expired as well as assess how much of that food is left in the container.

There is a need for a means to sense the amount and quality of a stock of ingredients automatically and remotely.

The object of the present invention is to overcome or at least substantially ameliorate the aforementioned problems.

### SUMMARY OF THE INVENTION

According to a first aspect of the present invention, there is provided a system for allowing a user to automatically measure the contents in a container, the system comprising:
(a) a base for holding the contents within the container;
(b) a lid for the base of the container;
(c) a central processing unit within the container;
(d) an antenna connected to the central processing unit for transmitting data;
(e) a software application running on a mobile device for communicating with the central processing unit;
(f) a remote server for connecting to the central processing unit and to the internet; and
(g) a sensor device connected to the central processing unit to create data files about the container;
   wherein the sensor device has a particle detector.

According to a second aspect of the present invention there is provided a container for allowing a user to automatically measure the contents in the container, the container comprising:
(a) a base for holding the contents;
(b) a lid for the base;
(c) a central processing unit in the container;
(d) an antenna for transmitting data;
(e) a software application running on a mobile device for communicating with the central processing unit; and
(f) a sensor device connected to the central processing unit to create data files about the container,
   wherein the sensor device has a particle detector.

The ‘contents’ may be food, drink, or other items.

The particle detector may include a gas sensor. Preferably the particle detector is removable. The particle detector may draw power from an internal battery.

Preferably, the central processing unit is programmed with the gas profiles of various different consumables and gases indicating expired consumables. The software application may allow a user to identify the contents of the container via manual input or automatically via a barcode reader program using the mobile device of the user. The barcode reader program in the software application may log products without needing to put those products into the container.

The sensor device may include a laser. The time of flight of the laser beam may determine the depth of the container and thereby the volume or amount of contents within it.

The laser may be used to ablate the contents so that a particle detector in the container can detect whether the contents have perished. Preferably, the particle detector is located within the lid of the container.

The sensor device may be manually activated using the software application on the mobile device, or may be automatically activated by the central processing unit.

The software application running on the mobile device with a camera may include a barcode reader program that can be used to identify the product put into the container by the user. The data regarding the product can be downloaded via the remote server and transmitted to the central processing unit via the antenna.

The container may include a weight scale.

The software application may create a shopping list for the user based on the contents automatically measured by the container. The software application may also recommend to a user that they consume the contents because they are about to expire. The software application may suggest meal recipes based on the remaining contents in the container. The software application may tell a user how much of any particular item is left within the container. The software application may be connected to a communication device such as a Google Home unit, or an Alexa Home unit, so that the communication device can be used as a means of making queries about the contents of the containers. The software application may be programmed to order new contents (including consumable items such as food and drink) based on the content levels in the containers. The software application may be programmed to provide recipe suggestions based on the products in the container. The software application may be programmed to provide nutritional information about the products in the container.

The system of the invention may also include a separate scale connected to the software application for weighing products which are not capable of being put into the container.

Any of the features described herein can be combined in any combination with any one or more of the other features described herein within the scope of the invention.

### BRIEF DESCRIPTION OF DRAWINGS

Embodiments of the invention will be described with reference to the following drawings, in which:

FIG. 1 is an illustration of a container according to an embodiment of the present invention for automatically measuring the amount and quality of consumable food or drink in the container.

FIG. 2 is an exploded view of the lid of the container of FIG. 1.

FIG. 3 is a perspective view of the underside of the lid of the container of FIGS. 1 and 2, showing a sensor device for the contents of the container.

FIG. 4 is an exploded view of the sensor device of FIG. 3.

FIG. 5 is a perspective view of the container of FIG. 1 showing the weight scale in the base of the container.

FIG. 6 is a perspective view of the container of FIG. 1 wherein the circuit board of the weight scale in the base of the container has been exposed.

FIG. 7 is an exploded view of the container of FIG. 1 showing the weight scale base separated from the body of the container.

FIG. 8 is another embodiment of a container according to the present invention featuring a separate scale for measuring the amount of food.

### DETAILED DESCRIPTION

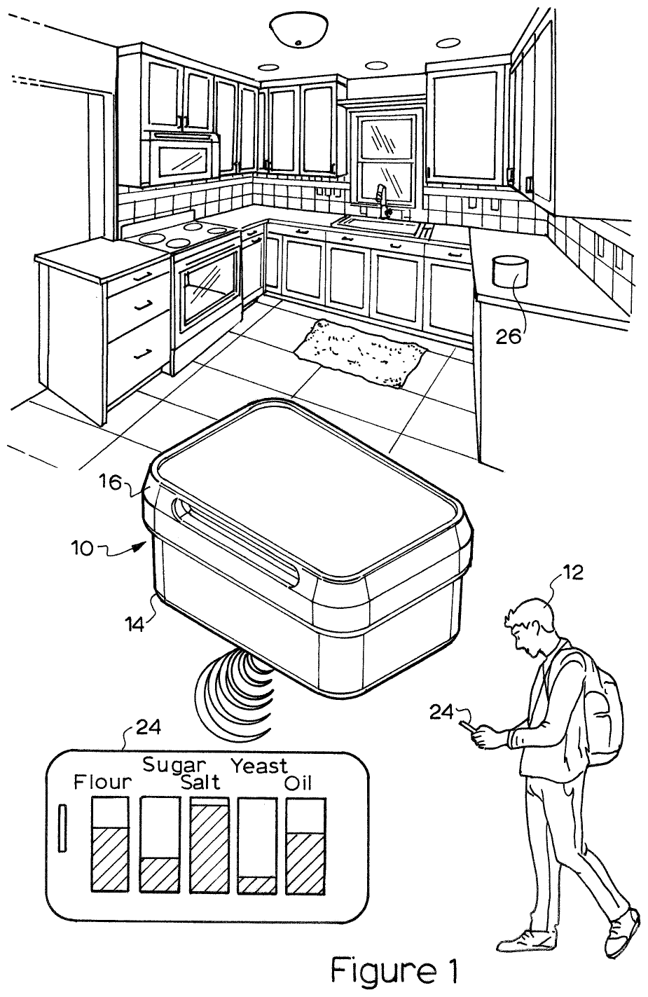

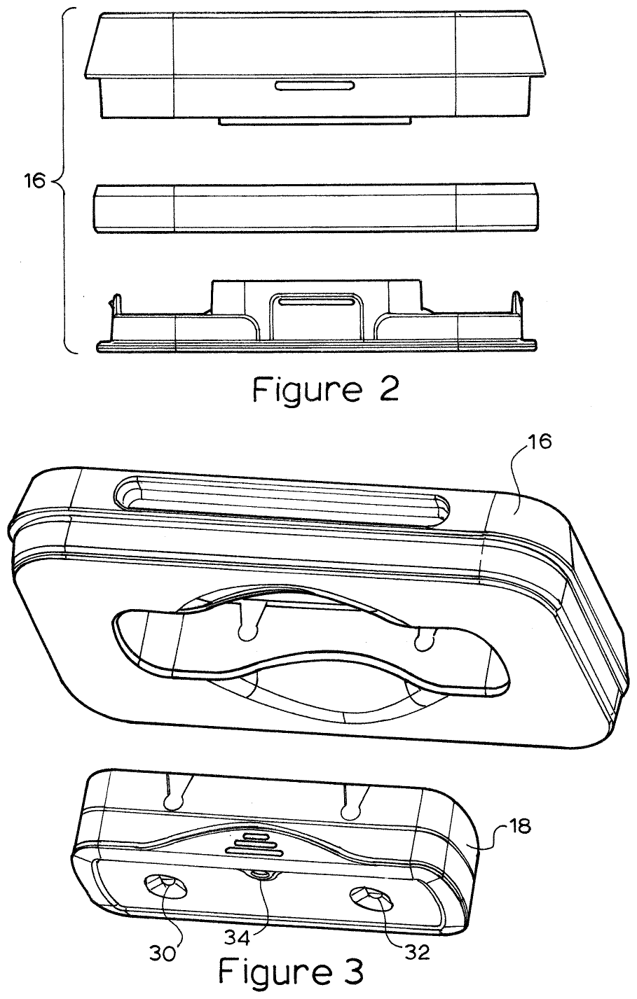

FIG. 1 shows a container 10 which allows a user 12 to automatically measure the amount and quality of the contents of the container 10. In most cases, the contents of the container will be food, but may also include drink or other items. The shape of the container 10 is depicted as a rectangular prism, but it could take any form, for example a cylinder in the case of drinks.

The container 10 has a body 14 for holding the contents (not shown), a lid 16 for sealing the container and a base 15 for the body 14.

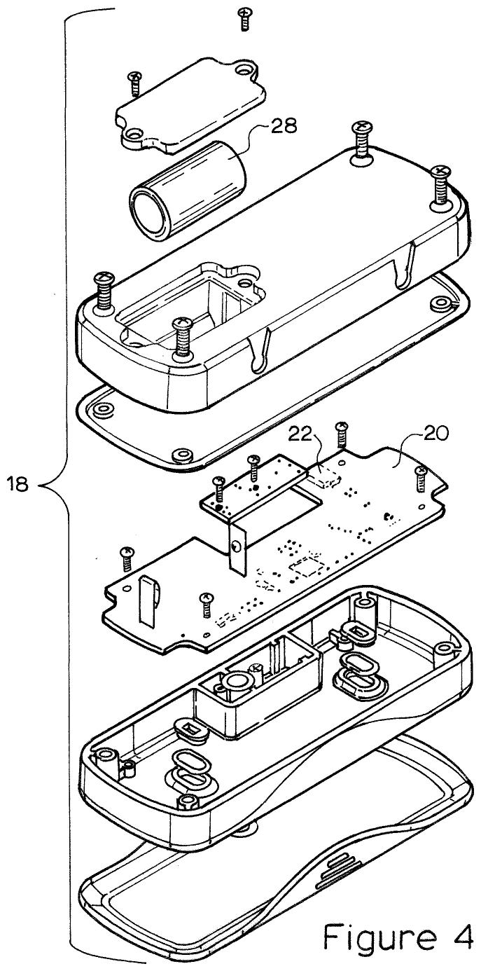

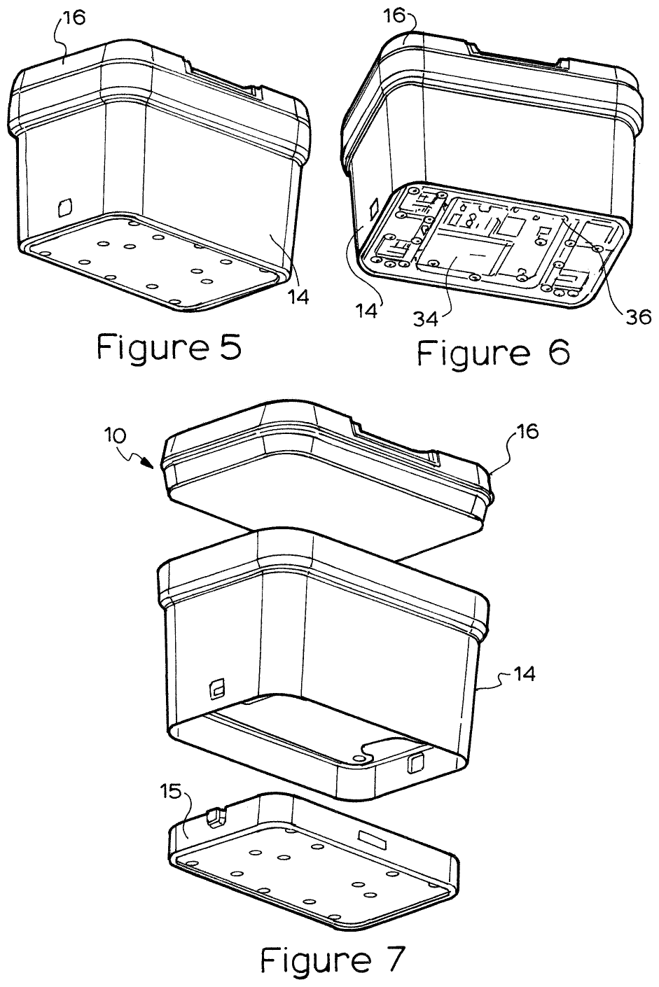

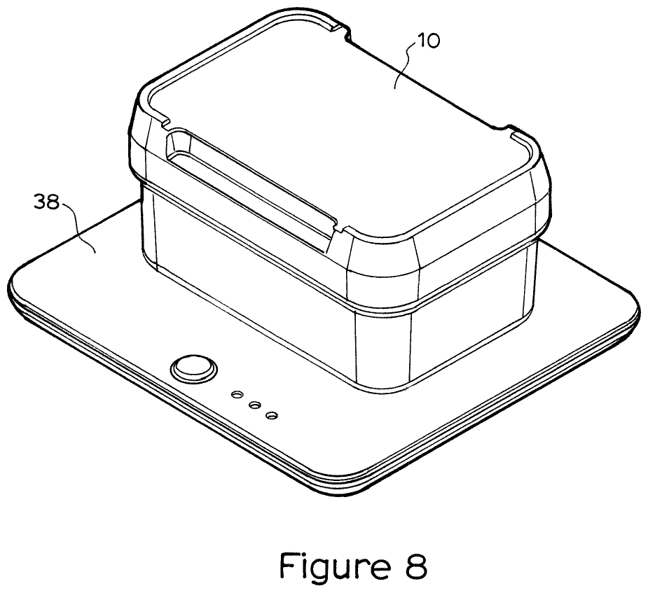

The container 10 has a sensor device 18 which is detachably connected within the lid 16. The sensor device 18 contains a central processing unit 20 (see FIG. 4) and an antenna 22. This enables the central processing unit 20 to communicate with a mobile device 24 (see FIG. 1) running a software application of the present invention and/or a remote server 26 so that the central processing unit can independently access the internet. The sensor device 18 is powered by an internal battery 28.

The mobile device 24 has a camera which can be used by the software application to read barcodes that feeds data about the products. The data regarding the product can be downloaded and transmitted to the central processing unit 20 via the antenna 22. The download can occur via the remote server 26 or via the software application on the mobile device 24 of the user 12. However, it is preferred that the download occurs via the remote server 26 so as not to rely on the mobile device 24 of the user 12 which may or may not be within the range of the container 10. In many cases, the user 12 will want to check the status of the contents of the container 10 whilst away from the home and outside the range of Bluetooth connectivity between the mobile device 24 and the container 10.

The sensor device 18 has lasers 30 and 32 to measure the depth of the container 10 and create a data file about the amount of food left in the container and then transmit that data file to the mobile device 24 and/or the central processing unit 20. The central processing unit 20 can use one laser if the other is temporarily dysfunctional or take the average reading from both lasers 30 and 32.

The sensor device 18 also has a particle detector 34. The particle detector 34 measures the gas within the container.

The particle detector 34 relays information to the central processing unit 20 which has profiles of different gases. For example, if the particle detector detects sulphur gas, then the sensor device will know that the food or drink within the container has gone off. The central processing unit 20 is programmed with the gas profiles of various other poisonous/toxic gases.

The central processing unit is programmed with the gas profiles of many foods. For example, chocolate has a particular gas profile, which can be sensed by the sensor device. If the central processing unit cannot recognise the gas profile of the contents, then it will ask the user 12 via the software application on the mobile device 24 to identify the contents.

The software application has a bar code scanner program (utilised via the camera on the mobile device 24 of the user 12) to identify products. Various other data about the contents based on the barcode can be downloaded via the remote server 26 to the central processing unit 20. This data may include average expiry of various consumables, such as milk.

The sensor device 18 also includes a thermometer, so that the data regarding the temperature (namely, the refrigeration) of the contents of the container 10 can be taken into account. The temperature data can be presented to the user 12 via the software application running on their mobile device 24.

The lasers 30 and 32 are capable of ablating food in the container 10 so that the particle detector 26 in the container 10. The particle detector 26 can measure the gas emitted from the ablated food as it spoils and thereby becomes a more reliable means of detecting the quality of the food than a mere use-by date label. In some cases, food can spoil before it's use-by date because it has been overheated. In other cases, food can remain fresh beyond its use-by date if it has been frozen.

The sensor device 18 can be activated via the software application, but it also periodically and automatically takes readings of the contents of the container 10 as directed by the central processing unit 20.

The container 10 has internal scales 34 controlled by a circuit board 36 in the base 15 to measure the weight of the contents of the container 10. The circuit board 36 communicates with central processing unit 20 via the antenna 22 and the remote server 26.

Another embodiment of the invention has an external scale 38, as shown in FIG. 8.

The mobile device 24 has a software application adapted to create a shopping list for the user 24 based on the amount of food automatically assessed by the container 10. The software application can suggest meal recipes based on the remaining stock levels of food. The software application can also issue notifications to the user via the mobile device 20 to quickly consume the food because it is going off. The software application can tell a user how much of an item is left within the container 10. The software application can be connected to a communication device such as a Google Home unit, or an Alexa Home unit, so that the communication device can be used as a means of making queries about the contents of the containers. The software application can also be programmed to order new contents (including consumable items such as food and drink) based on the content levels in the container 10.

Thereby, the container of the present invention provide a means for automatically and remotely sensing the amount and freshness of the contents of the container.

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

1. A system for allowing a user to automatically measure the contents in a container, the system comprising:
(a) a base for holding the contents within the container;
(b) a lid for the base of the container;
(c) a central processing unit within the container;
(d) an antenna connected to the central processing unit for transmitting data;
(e) a software application running on a mobile device for communicating with the central processing unit;
(f) a remote server for connecting to the central processing unit and to the internet; and
(g) a sensor device connected to the central processing unit to create data files about the container;
wherein the sensor device has a particle detector for measuring the contents of the container, and the sensor device includes a laser to ablate the contents of the container to assist the particle detector.

2. (canceled)

3. (canceled)

4. The system of claim 1, wherein the laser is used as a time-of-flight sensor to determine the height of the contents within the container.

5-14. (canceled)
